(19) United States
(12) Patent Application Publication
(10) Pub. No.: US 2019/0060577 A1
(43) Pub. Date: Feb. 28, 2019

(54) SMART SYRINGE

(71) Applicant: Cypress Semiconductor Corporation, San Jose, CA (US)

(72) Inventor: Darrin T. Vallis, Austin, TX (US)

(73) Assignee: Cypress Semiconductor Corporation, San Jose, CA (US)

(21) Appl. No.: 15/851,062

(22) Filed: Dec. 21, 2017

Related U.S. Application Data
(60) Provisional application No. 62/552,578, filed on Aug. 31, 2017.

(51) Int. Cl. A61M 5/315; G01F 23/26; G01B 7/14

(52) U.S. Cl. CPC A61M 5/31568; G01F 23/26; G01B 7/14; A61M 2205/3553; A61M 2205/3317; A61M 2205/3584; A61M 2205/702; A61M 2205/502; A61M 2205/609; A61M 2205/3368; A61M 2205/8206; A61M 2205/587; A61M 2205/3327

(57) ABSTRACT

A smart syringe is described that is capable of measuring a position of a plunger within the barrel of the smart syringe and of communicating the status of a liquid pharmaceutical within the smart syringe to a patient database.

### RELATED APPLICATIONS

This patent application claims the benefit of U.S. Provisional Patent Application No. 62/552,578 filed Aug. 31, 2017, which is incorporated by reference herein in their entirety.

### TECHNICAL FIELD

The present disclosure relates generally to drug delivery mechanism, and more particularly syringes with measurement and communication abilities.

### DESCRIPTION OF THE DRAWINGS

FIG. 1 illustrates smart syringe, according to one embodiment.

FIG. 2A illustrates a plunger of a smart syringe, according to one embodiment.

FIG. 2B illustrates an inductive sensor coil of a smart syringe according to one embodiment.

FIG. 3 illustrates various embodiments of metallization patterns of a label on a smart syringe.

FIG. 4A illustrates a smart syringe with a plunger at a first position, according to one embodiment.

FIG. 4B illustrates a smart syringe with a plunger at a second position, according to one embodiment.

FIG. 4C illustrates a smart syringe with a plunger at a third position, according to one embodiment.

FIG. 4D illustrates a smart syringe with a plunger at a fourth position, according to one embodiment.

FIG. 5A illustrates a controller circuit coupled to an inductive sensor coil and an antenna, according to one embodiment.

FIG. 6A illustrates a plunger, piston, and applicator of a smart syringe according to one embodiment.

FIG. 6B illustrates various embodiments of an applicator with detection and feedback mechanisms.

FIG. 7 a system including a smart syringe, according to one embodiment.

FIG. 8 is a method for measuring a liquid in a smart syringe, according to one embodiment.

### SUMMARY

In the first embodiment, the present invention is directed to an apparatus, such as a syringe, for dispensing a liquid pharmaceutical. The syringe may include an electrode or conductive material disposed on the barrel of the syringe and a second electrode disposed on the plunger. The second electrode may be configured to measure a physical or electrical property, such as inductance or capacitance, which may be altered by the first electrode. The syringe may also include a wireless communication circuit that communicates with a database and which is operable to communicate a status of the liquid pharmaceutical. As the second electrode moves relative to the first electrode, a linear position along the syringe may be calculated and converted into information pertaining to the liquid pharmaceutical and its delivery. In the first embodiment, the measurement circuit and the wireless communication may be disposed on the same integrate circuit (IC) or on separate ICs. The IC (or ICs) may be disposed with n applicator of the syringe, which may be positions at one end of the piston opposite to the plunger. The IC (or ICs) may be coupled to the second electrode by a sensor interconnect dispose along, on, near, or in the piston. Display mechanisms and detection apparati may also be disposed in the applicator to detect a user and provide information on the contents of the syringe.

A second embodiment of the present invention is a method for measuring a volume of disposed liquid, such as a pharmaceutical, that includes measuring an electrical property of a first electrode that may be altered by the proximity of the first electrode to a second electrode. The electrical property may be converted to a linear distance value, which may be in turn converted to a volume. The volume, or some representation of that volume, may be communicated to a host or a database through a wireless protocol. The first and second electrodes may be disposed on the plunger and the barrel of a syringe and may be configured as an inductance sensor or a capacitance sensor, or any other sensor capable of measuring a change in electrical or physical properties of at least one the electrodes.

A third embodiment of the present invention may be a smart syringe including electrodes for measuring a linear displacement of a plunger within a barrel of the syringe and for displaying that information to a user, communicating the information to a host, and storing the information in a database. The smart syringe may further include sensing and display circuitry for an interface with a user.

### DETAILED DESCRIPTION

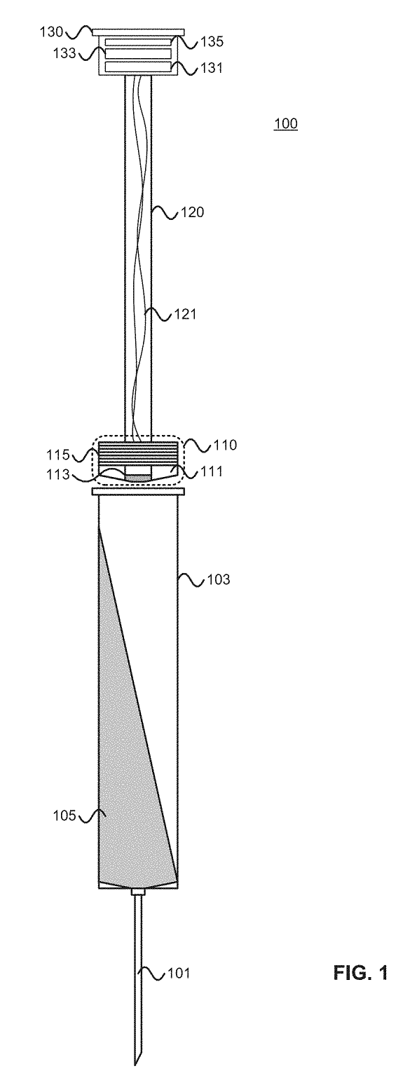

FIG. 1 illustrates one embodiment of a syringe 100 capable of measuring and communicating drug dosage. Syringe 100 may include a needle 101 attached to a barrel 103 into which plunger 110 is inserted by piston 120 when force is applied to applicator 130. Label 105 may be applied to barrel 103 with an adhesive. Label 105 may include metallic elements and cover portions of barrel 103 progressively. That is, barrel 103 may be covered with metal to a greater extent at one end than it is at the other. In one embodiment, label 105 may be applied with an adhesive. In another embodiment, label 105 and the metallic properties thereof may be printed directly on barrel 103, such that a separate adhesive is not used. In still another embodiment, metal materials may be integrated into barrel 103 itself.

Plunger 110 may include plunger seal 111 for ensuring the liquid contained within barrel 103 does not exit or enter except through needle 101. Plunger 110 may also include a temperature sensor 113 for measuring the temperature of a liquid in barrel 103. Additionally, plunger 110 may include an inductive sensor 115, comprising a number of wire coils. Inductive sensor 115 and temperature sensor 113 may be coupled to a controller 133 in applicator 130 by sensor interconnect 121 disposed within or along piston 120. Sensor interconnect 121 may, in various embodiments, be constructed of discrete wires, a flexible printed circuit board (PCB), conductive materials printed on piston 120 directly, or a conductive plastic molded into the structure of piston 120. One of ordinary skill in the art would understand that any method for galvanic communication of an electrical signal may be used to couple inductive sensor 115 and temperature sensor 113 to controller 133. Controller 133 may be coupled to a power source, such as battery 131, and an antenna 135, both of which may be disposed within applicator 130 in one embodiment. In some embodiments, controller 133 may be coupled to circuit elements for providing audible, visual, or tactile feedback to a user or patient. Such embodiments may include, but are not limited to, light emitting diodes (LEDs) and piezoelectric speakers or actuators.

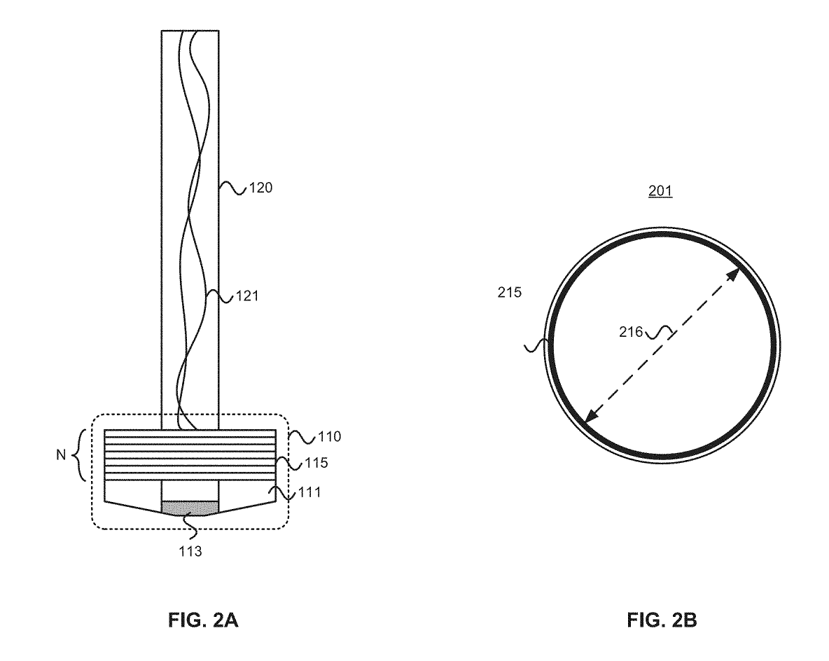

FIG. 2A illustrates plunger 110 and piston 120 from FIG. 1. Inductive sensor 115 may include a number, N, of coil turns that yields a coil length CL. FIG. 2B illustrates a cross-sectional view 201 of plunger 110, such that the inductive sensor 115 of FIGS. 1 and 2A is shown as a ring. Inductive sensor 115 may be constructed of coils of an insulated wire 215 having a diameter 216. The coils of insulated wire 215 comprising inductive sensor 115 may be wound such that the plane formed by the coils is perpendicular to the line formed by plunger 110 or piston 120. Inductance of the coils of inductive sensor 115 may be given by

$$L = \frac{( {D}^{2} + {N}^{2} )}{18 D + 40 CL} ,$$

where D is the diameter of the coils, N is the number of coils, and CL is the coil length, or the length of the wire with which the inductive sensor is constructed.

In one embodiment, the inductance of inductive sensor 115 is matched with inductive sensing circuitry in controller 133. The goal of the matching is to generate sufficient signal in the presence of the metal in label 105. A good rule of thumb is to achieve a signal-to-noise ratio (SNR) of 100:1. An SNR of 100:1 provides accurate linear motion sensing as the wire coil of inductive sensor 115 moves through barrel 103 and interacts more or less with the metal of label 105.

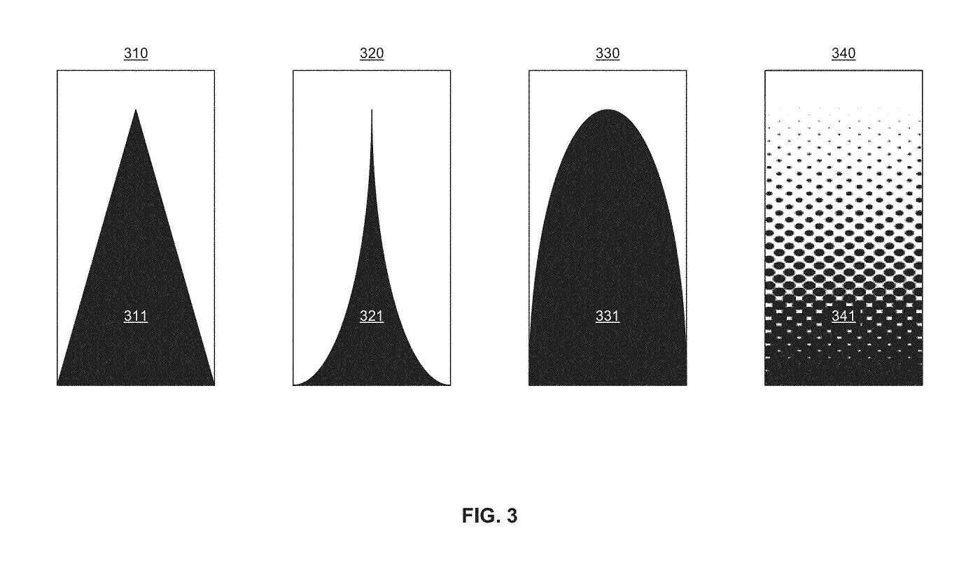

FIG. 3 illustrates various embodiments of metallized patterns which may be adhered to barrel 105 with label 103, or otherwise disposed on a surface of barrel 103. Label 310 may include a linear, triangular metal pattern 311. In this embodiment, the measured change in inductance may be linear as plunger 110 moves through barrel 103. Labels 320 and 330 may include non-linear patterns metal patterns 321 and 331. As plunger 110 moves through barrel 103, onto which labels 320 and 330 are adhered, the inductance measured by controller 133 may change more rapidly or more slowly in various positions within barrel 105. In these embodiments, the patterns on label 105 may be optimized to provide greater positional resolution at the start of drug delivery or the end of drug delivery. In other embodiments, greater position resolution may be provided in the middle of the barrel with different patterns. In still another embodiment, a discontinuous pattern 341 may be disposed with label 340.

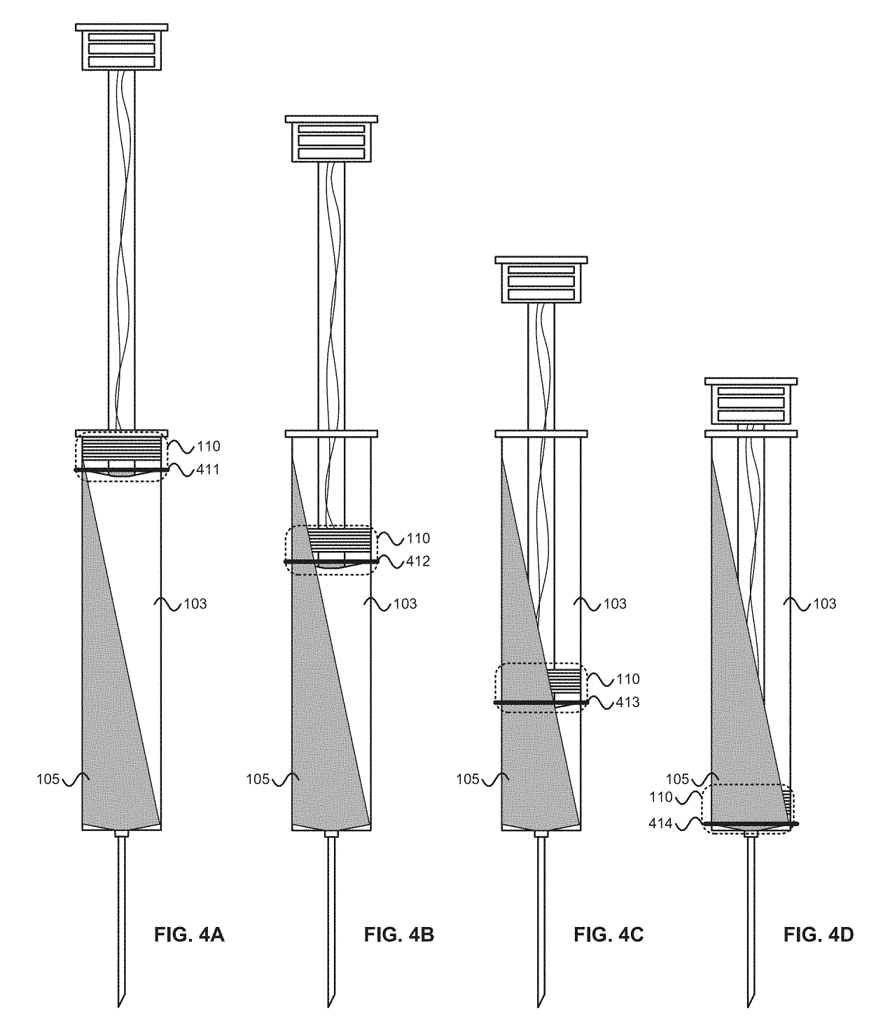

FIGS. 4A-4D illustrate various positions of plunger 110 as it travels through barrel 105 with pressure applied to applicator 130. Controller 133 may measure a change in the magnetic field of inductive sensor 115 as it interacts with more or less metalized portions of label 105. The change in magnetic field may be detected as a chance in inductance of the inductive sensor. Controller 133 may then convert the measured change in the magnetic field to a linear position of plunger 110 within barrel 103. The position of plunger 110 within barrel 103 may be used to calculate the volume of liquid and the amount of drug delivered by syringe 100. In other embodiments, the position of plunger 110 within barrel 103 may be used to calculate the volume of liquid that is withdrawn into the syringe.

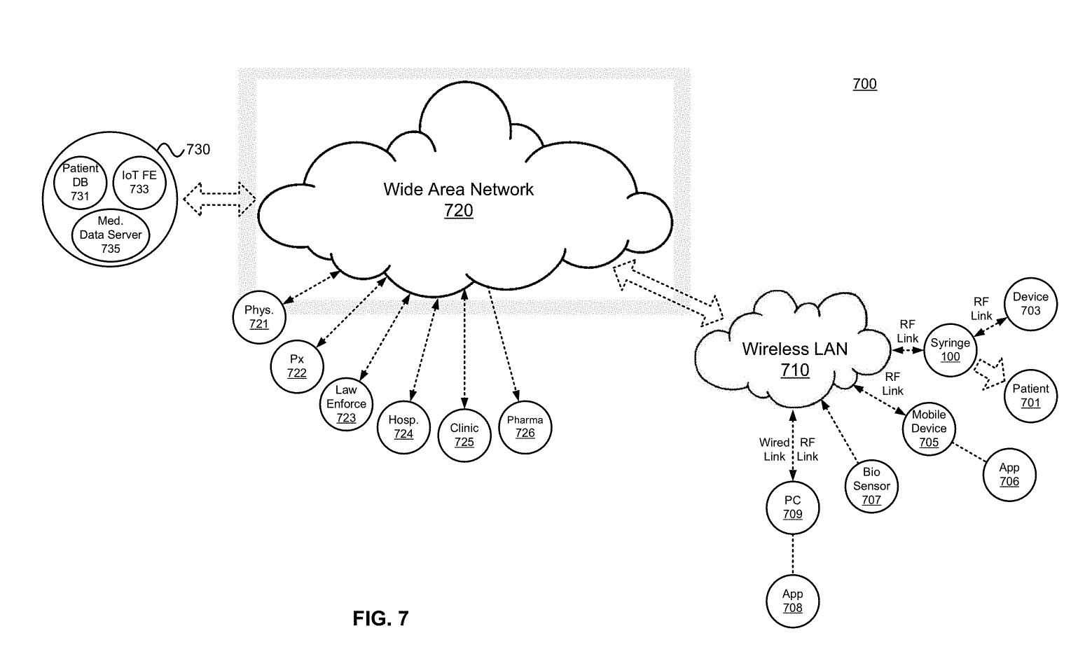

FIG. 4A illustrates a first position 411, whereat plunger 110 does not overlap any of the metallization of label 105. Position 411 may correspond to an initial position set when the syringe is filled with a liquid. This position may be set by the manufacturer when the syringe is prepared. In other words, this maybe the “as shipped” condition, for which no liquid or drug has been dispensed. In another embodiment, this position may be reset when the syringe is filled with a liquid during treatment. In this embodiment, the syringe may be filled with a liquid by a patient or healthcare provider. The ending position of the plunger may be entered with an input on applicator 130 to alert a device that is coupled to the smart syringe and to a patient database that a volume of liquid now fills the syringe. The amount of liquid (or drug) that is then dispensed may be recorded. See FIG. 7 for discussion of the communication of drug delivery and patient information.

FIG. 4B illustrates a second position 412, wherein plunger 110 overlaps a portion of the metallization of label 105 corresponding a position of plunger 110 within barrel 103. In one embodiment, the position of plunger 110 within barrel 103 may correspond to a volume of liquid that has been dispensed through needle 101. The volume of liquid may be derived from the circumference of the barrel 103 and the distance traveled by the plunger 110. This volume may be calculated by controller 133 based on the geometry of barrel 103, it may be derived from a look-up table (LUT) with volumes at various positions, or it may be calculated by a host in communication with controller 133. In this embodiment, the position of plunger 110 within the barrel 103 may be sent to a host, which calculates the volume of liquid delivered or remaining and returns that value to controller 133. In another embodiment, the position of plunger 110 in FIG. 4B may be a starting position, whereat the plunger 110 is not initially withdrawn beyond the metallization of label 105.

FIG. 4C illustrates a third position 413, wherein plunger 110 overlaps a portion of the metallization of label 105 corresponding a position of plunger 110 within barrel 105. In one embodiment, the position of plunger 110 within barrel 103 may correspond to a volume of liquid that has been dispensed through needle 101. The volume of liquid may be derived from the circumference of the barrel 103 and the distance traveled by the plunger 110. In another embodiment, the position of plunger 110 in FIG. 4B may be a starting position, whereat the plunger 110 is not initially withdrawn beyond the metallization of label 105.

FIG. 4D illustrates a fourth position 414, whereat plunger 110 overlaps a portion of the metallization of label 105 corresponding to a position of plunger 110 that is depressed as far as possible within barrel 103. Position 413 may correspond to complete delivery of the liquid that filled barrel 103. In another embodiment, position 413 may correspond to a starting location as a volume of liquid is withdrawn from a receptacle or a patient. As plunger 110 moves away from position 413, a volume of liquid may be measured.

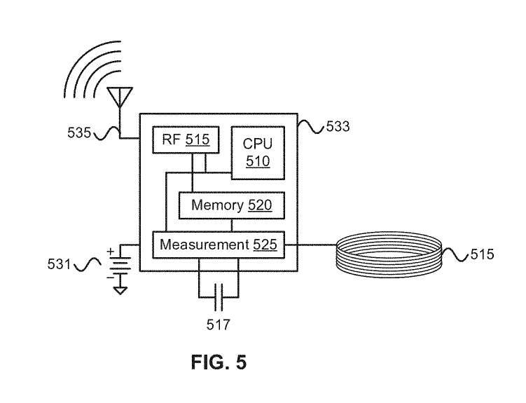

FIG. 5A illustrates an embodiment of a controller (IC) 533 capable of measuring an inductance on a coil 515 or a capacitance on a capacitance sensor 517 and communicating wirelessly to an external device through antenna 535. Battery 531 may provide power to IC 533. In one embodiment, battery 533 is a battery. In another embodiment, battery 533 is a super-cap. IC 533 may include an RF circuit 515 coupled to antenna 535 as well as CPU 510 and memory 520. RF circuit 515 and antenna 535 may be used to communicate information to and from memory 520 or CPU 510. CPU 510 may send information or commands to RF circuit 515 and memory 520 based on the received information from the wireless connection. CPU 510 may also be coupled to a measurement circuit 525 for measuring inductance on coil 515 or capacitance on capacitance sensor 517. In one embodiment, capacitance sensor 517 may include coil 515 for either self capacitance or mutual capacitance measurement. Measurement circuit 525 may include separate measurement circuitry for capacitance measurement and inductance measurement. Measurement circuit 525 may also use the same measurement circuitry, but configured differently for capacitance measurement and inductance measurement.

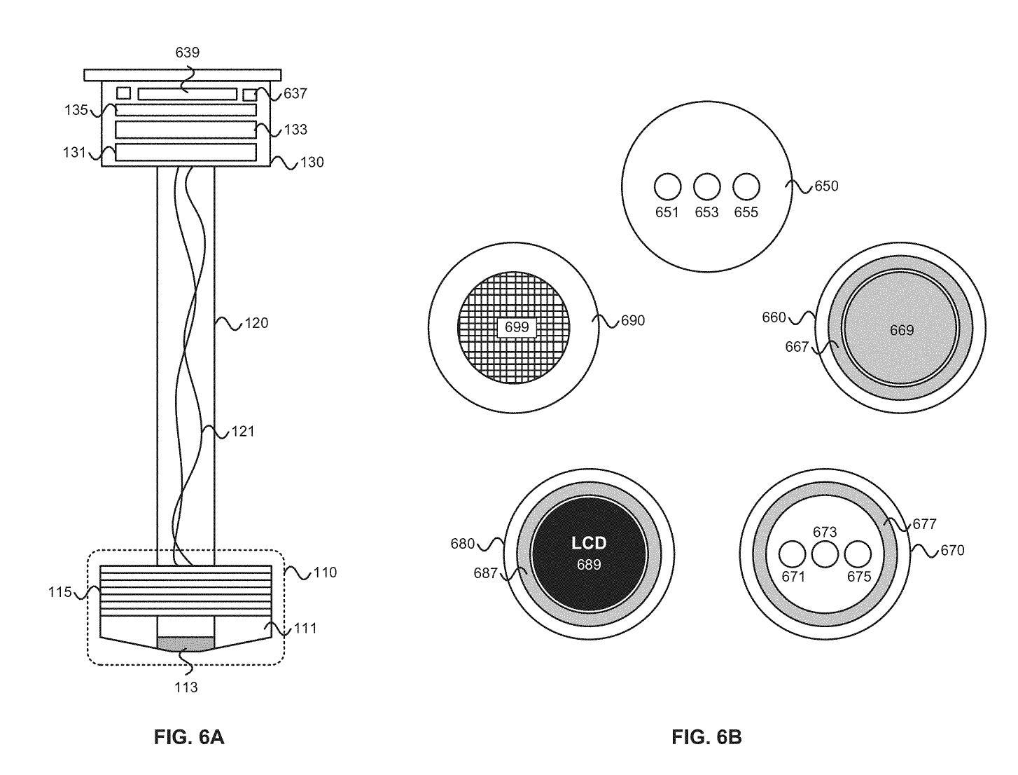

FIG. 6A illustrates one embodiment of a plunger, piston, and control module as illustrated in FIG. 1, but further illustrating a display mechanism 639 and detection mechanism 637. The control module 610 may include controller 133, antenna 135, and battery 131 as shown in FIG. 1. However, additional circuit elements may also be coupled to controller 133. These additional elements may include sensors, such as capacitance sensor or fingerprint sensor or accelerometer, display units, such as graphical displays (i.e., LCDs) or light emitting diodes (LEDs), and tactile or auditory response mechanisms such as piezoelectric speakers. Capacitance sensors and accelerometers may be used for wake-up functions, such that the inductive sensor and communication circuits of the smart syringe are not active unless and until the capacitive sensor or sensors are activated. Capacitance sensors may be implemented as self capacitance sensors, where a single electrode is used, or mutual capacitance sensors, where at least two electrodes are coupled to a capacitance measurement circuit. In the case of an accelerometer, detection of movement may have a “wake-on-movement” function so that a syringe that is stored is not consuming power and is only activated once picked up by a patient or care-giver. A fingerprint sensor may be used to determine who administers the drug.

Display units such as an LED or an LCD may alert the patient or caregiver as to the status of the drug contained within the barrel of the syringe. In one embodiment, display units and detection units may both be implemented, such that a capacitance sensor is disposed around or near an LED or LCD.

FIG. 6B illustrates various embodiments of applicators with additional functionality for detection and feedback. Applicator 650 may include a number of LEDs 651, 653, and 655. Each LED may illuminate for various reasons. In various embodiments, LEDs 651, 653, and 655 may illuminate to alert a user that the temperature of the drug has exceeded the allowed range, that the smart syringe is paired with a receiver device, that the drug has been dispensed property or improperly, or another response, alert, or notification.

Applicator 660 may include a capacitance sensor with two sensing electrodes 667 and 669. Electrodes 667 and 669 may be configured to measure either self capacitance or mutual capacitance or both. The presence of a conductive object on the capacitance sensor formed by at least one of the electrodes 667 and 669 may cause the controller (133 of FIGS. 1 and 6A) to attempt communication with a wireless device. Such a feature may allow for “wake-on-touch,” such that the controller is not communicating to a wireless device unnecessarily, thus preserving battery.

Applicator 670 may include a capacitance sensor, formed by electrode 677, and LEDs 671, 673, and 675 so that the functionality of both applicator 650 and 660 may be realized. While only one electrode is illustrated, it may be desirable to have more than one sensing electrode for mutual capacitance sensing or to poll the controller to measure various properties of the drug (temperature, volume, etc.) and alert the user to the status through LEDs 671, 673, or 675.

Applicator 680 may include a capacitance sensor, formed by electrode 687, and an LCD 689. In this embodiment, the functionality of applicator 660 may be realized, as well as additional display functionality enabled by LCD 689. Rather than mere yes/no/maybe responses that are possible with LEDs, Applicator 680 may provide detailed information on LCD 689 about the temperature of the drug, the volume that remains, the wireless connection status, as well as drug information that may be programmed into the syringe by the manufacturer or communicated to the controller through the wireless connection.

Applicator 690 may include a fingerprint sensor 699. Fingerprint sensor 699 may allow for controller 133 to communicate the identification of the person using the syringe, if the identity is stored within the controller or a connected memory. In one embodiment, the controller may send a fingerprint image to a database to compare to a set of known fingerprints. In one embodiment, if a fingerprint is read that is not allowed, the fingerprint information as well as the drug information may be transmitted to law enforcement, to the desired patient, or to healthcare providers.

The embodiments of FIG. 6B are separate for ease of explanation. However, one of ordinary skill in the art would understand that the features of the various embodiments may be combined to provide a more feature-rich smart syringe. For example, it may be desirable to combine all of the features: LEDs, capacitance sensing, an LCD, and a fingerprint sensor into a single applicator. This embodiment is bound only by the sterics of so many interface devices disposed in a small space.

FIG. 7 illustrates a system 700 into which a smart syringe 100 may be integrated. Smart syringe 100 may be held by or used to treat patient 701. Patient 701 may be holding or otherwise be equipped with a device 703 with a wireless ID, which may be wirelessly connected to smart syringe 100 through a radio frequency (RF) link. Device 703 or smart syringe 100 may be connected to a wireless local area network (LAN) through an RF link. A mobile device 705 (e.g. a cell phone) or a personal computer (PC) 709 may run patient applications 706 or 708, respectively, to access a patient database 731 through wireless LAN 710 and wide area network 720. Patient applications 706 and 708 may display historical information regarding the drugs delivered to patient 701. Patient applications 706 and 708 may modify recommended dosages based on expert system algorithms in one embodiment. In another embodiment, recommended dosages may be pushed to patient applications 706 or 708 by medical professionals upon receipt of drug delivery and health information from patient 701. In one embodiment, bio sensors 707 may measure and record information from patient 701 and communicate that information to the patient database 731, PC 709, or mobile device 705. Information from bio sensors 707 may be used to modify or confirm recommended dosages. In another embodiment, a dedicated device may receive information from bio sensors 707. In this embodiment the dedicated device may be device 703 which corresponds to patient 701. In another embodiment, the dedicated device may be separate from device 703.

Wireless LAN 710 may be connected to wide area network 720. Also connected to wide area network may be computing devices that area accessible to physicians 721, pharmacies 722, law enforcement offices 723, hospitals 724, health clinics 725, and pharmaceutical companies 726. Each of these entities may be able to access information from smart syringe 100, the various sensors and applications, or patient database 731 through wide area network 720.

Patient database 731 may be one of several databases located on servers 730. Servers 730 may also include an internet-of-things (IoT) front end 733 for supporting the connectivity of the various devices that may access patient information through wide area network 720 and the wireless LAN 710. Servers 730 may also include a medical data server 735 for storing information related to drugs and other elements of patient care. Medical data server may be used by the various healthcare providers and device applications to keep up-to-date on drug facts, recommendations, and literature.

Physicians 721, pharmacies 722, hospitals 724, and clinics 725 may notify patients to administer drugs from syringe 100 based on the time day, notes from a medical service provider, or data from bio sensors 707. Pharmaceutical companies 726 or health care providers may alert patients to drug information based on readings from temperature sensor 113. That is, if syringe 100 reaches a temperature that is outside the allowable range for safe storage of the drug stored within syringe 100, a patient may be notified that the drug's safety or efficacy may be compromised.

Healthcare providers may send secure notifications to syringe 100 through wide area network 720 and wireless LAN 710 approving dispensation of a drug. If syringe 100 notifies the healthcare provider that a non-approved drug delivery occurs, the healthcare provider may notify law enforcement 723. In one embodiment, law enforcement 723 may be notified automatically.

A smart syringe may allow physicians, pharmacies, hospitals, clinics, or other healthcare providers to remotely monitory the date, time, amount, and time of drugs that are self-administered by patients. This remote monitoring increases the effectiveness of prescription drugs by ensuring the prescribed dosage and deliveries without requiring healthcare providers to be physically with the patient at the time of delivery or to “spot check” patient self-care with house calls or patient visits to the healthcare facility.

A smart syringe may allow pharmacies to notify patients when a prescription is in need of a refill based on data from the syringe or multiple syringes. Prescriptions may be automatically refilled based on information received by the pharmacy.

A smart syringe may reduce adverse drug effects (ADEs) that may be realized in a hospital by pairing the smart syringe with a patient specific device (e.g. device 703 of FIG. 7). The smart syringe may communication wireless with the patient specific device to verify the patient's identity. As the smart syringe is pre-programmed and loaded with the drug, the smart syringe may communicate with a medical data server to validate the drug and dosage. That is, the syringe may initiate a verification that the drug and dosage contained therein is prescribed to the patient. The smart syringe may communication a validation to a nurse or other healthcare professional that delivery of the drug is prescribed.

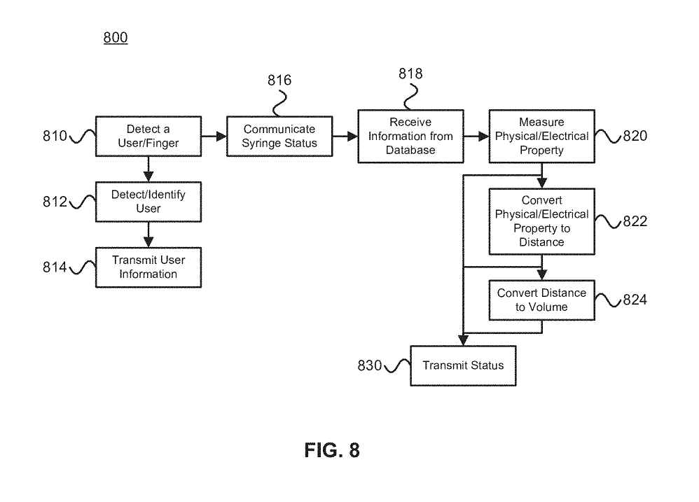

FIG. 8 illustrates a method 800 for using a smart syringe according to one embodiment. A finger may be detected on the applicator on step 810. This may be done with a fingerprint sensor, a capacitance sensor, or some other device for detecting the presence of a user. In another embodiment, an accelerator may be used to detect that the smart syringe may be used and to trigger an interrupt to wake up the controller for communication and measurement. In one embodiment, the user's finger may be detected and imaged by a fingerprint sensor in step 812 and the identity of that user transmitted to a host and a database in step 814. In one embodiment, the identity of the user may not be communicated until after the drug stored in the smart syringe is delivered. Also after a finger is detected, a status of the drug may be communicated to the host or to the user in step 816. The temperature, viability, shelf-life, or other parameters that may affect the efficacy of the drug may be communicated. These status updates may be self-contained with the smart syringe, or they may be derived from information that is communicated to the smart syringe over a wireless connection. In one embodiment, the status is communicated to the user through an interface on the syringe. In another, the RF circuit may be activated and status communicated to a host which may store information on a database, or to a wireless device that can display the necessary information to the user. Additional information may be received by the smart syringe in step 8818 through the wireless connection.

Method 800 may measure a physical or electrical property of a sensor in step 820. The physical property may be an inductance of a wire coil. The physical or electrical property may also be a capacitance sensor. The physical or electrical property may be converted to a linear distance in step 822 and the lineal distance converted to a volume in step 824. The physical or electrical property, the linear distance, or the volume may then be communicated in step 830.

While the above description is directed to a smart syringe, one of ordinary skill in the art could apply similar methods and apparati to the measurement of any precision linear sensor. The movement of a sensor over a relatively static element capable of changing the electrical or physical properties of the sensor may measured and converted to a distance, area, or volume in various embodiments.

In the above description, numerous details are set forth. It will be apparent, however, to one of ordinary skill in the art having the benefit of this disclosure, that embodiments of the present invention may be practiced without these specific details. In some instances, well-known structures and devices are shown in block diagram form, rather than in detail, in order to avoid obscuring the description.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared and otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “encrypting,” “decrypting,” “storing,” “providing,” “deriving,” “obtaining,” “receiving,” “authenticating,” “deleting,” “executing,” “requesting,” “communicating,” “initializing,” or the like, refer to the actions and processes of a computing system, or similar electronic computing device, that manipulates and transforms data represented as physical (e.g., electronic) quantities within the computing system's registers and memories into other data similarly represented as physical quantities within the computing system memories or registers or other such information storage, transmission or display devices.

The words “example” or “exemplary” are used herein to mean serving as an example, instance or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such.

Specific commands ore messages referenced in relation to the above-described protocol are intended to be illustrative only. One of ordinary skill in the art would understand that commands of different specific wording but similar function may be used and still fall within the ambit of the above description.

Embodiments described herein may also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer-readable storage medium, such as, but not limited to, any type of disk including floppy disks, optical disks, CD-ROMs and magnetic-optical disks, read-only memories (ROMs), random access memories (RAMs), EPROMs, EEPROMs, magnetic or optical cards, flash memory, or any type of media suitable for storing electronic instructions. The term “computer-readable storage medium” should be taken to include a single medium or multiple media (e.g., a centralized or distributed database and/or associated caches and servers) that store one or more sets of instructions. The term “computer-readable medium” shall also be taken to include any medium that is capable of storing, encoding or carrying a set of instructions for execution by the machine and that causes the machine to perform any one or more of the methodologies of the present embodiments. The term “computer-readable storage medium” shall accordingly be taken to include, but not be limited to, solid-state memories, optical media, magnetic media, any medium that is capable of storing a set of instructions for execution by the machine and that causes the machine to perform any one or more of the methodologies of the present embodiments.

The algorithms and displays presented or referenced herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the present embodiments are not described with reference to any particular programming language. It will be appreciated that a variety of programming languages may be used to implement the teachings of the embodiments as described herein.

The above description sets forth numerous specific details such as examples of specific systems, components, methods and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth above are merely exemplary. Particular implementations may vary from these exemplary details and still be contemplated to be within the scope of the present invention.

It is to be understood that the above description is intended to be illustrative and not restrictive. Many other embodiments will be apparent to those of skill in the art upon reading and understanding the above description. The scope of the invention should, therefore, be determined with reference to the appended claims, along with the full scope of equivalents to which such claims are entitled.

What is claimed is:

1. An apparatus for dispensing a liquid pharmaceutical comprising:
a first electrode disposed on a barrel of a syringe;
a second electrode disposed on a plunger of the syringe, wherein the second electrode is coupled to a measurement circuit disposed on an applicator of the plunger to measure an electrical property of the second electrode, the electrical property of the second electrode alterable by the first electrode;
a temperature sensor disposed on the plunger of the syringe, the temperature sensor coupled to the measurement circuit and for measuring a temperature of the liquid pharmaceutical; and
a wireless communication circuit disposed on the applicator of the plunger in operable communication with a database and configured to communicate a status of the liquid pharmaceutical.

2. The apparatus for dispensing a liquid pharmaceutical of claim 1, wherein the status is derived from a linear position of the second electrode along a length of the first electrode.

3. The apparatus for dispensing a liquid pharmaceutical of claim 1, wherein the electrical property of the second electrode is inductance and wherein the inductance of the second electrode increases and decreases in response to a position of the plunger within the barrel of the syringe.

4. The apparatus for dispensing a liquid pharmaceutical of claim 1, wherein the electrical property of the second electrode is capacitance and wherein the capacitance of the second electrode increases and decreases in response to the position of the plunger within the barrel of the syringe.

5. The apparatus for dispensing a liquid pharmaceutical of claim 1, wherein the measurement circuit and the wireless communication circuit are disposed on the same integrated circuit (IC).

6. The apparatus for dispensing a liquid pharmaceutical of claim 1, wherein the measurement circuit and the wireless communication circuit are disposed with an applicator connected to a piston, the piston connected to the plunger.

7. The apparatus for dispensing a liquid pharmaceutical of claim 1, wherein the second electrode is coupled to the measurement circuit by a sensor interconnect.

8. The apparatus for dispensing a liquid pharmaceutical of claim 7, wherein the sensor interconnect comprises at least one wire disposed within a piston between the plunger and an applicator.

9. The apparatus for dispensing a liquid pharmaceutical of claim 1, further comprising a user interface disposed on an applicator, the interface for communicating information about the liquid pharmaceutical to a user.

10. The apparatus for dispensing a liquid pharmaceutical of claim 9, wherein the user interface is an interface element selected from the group consisting of at least one LED, a capacitance sensor, a liquid crystal display (LCD), and a fingerprint sensor.

11. (canceled)

12. A method for measuring a dispensed liquid pharmaceutical comprising:
measuring an electrical property of a first electrode, the electrical property of the first electrode operatively altered by an overlap of the first electrode and a second electrode;
converting the measured electrical property to a linear value, the linear value representative of a position of a plunger within a barrel of a syringe;
converting the linear value to a volume of liquid pharmaceutical; and
communicating the volume of liquid pharmaceutical to a database over a wireless protocol.

13. The method for measuring a dispensed liquid pharmaceutical of claim 12, wherein:
the first electrode is disposed on the plunger; and
the second electrode is disposed on the barrel of the syringe.

14. The method of measuring a dispensed liquid pharmaceutical of claim 12, wherein the electrical property of the first electrode is inductance and wherein the inductance of the first electrode increases and decreases in response to the plunger's position within the barrel of the syringe.

15. The method of measuring a dispensed liquid pharmaceutical of claim 12, wherein the electrical property of the first electrode is capacitance and wherein the capacitance of the first electrode increases and decreases in response to the plunger's position within the barrel of the syringe.

16. A smart syringe comprising:
a first electrode disposed on a plunger of the smart syringe;
a second electrode disposed on a barrel of the smart syringe; and
a temperature sensor disposed on the plunger of the syringe, the temperature sensor coupled to the measurement circuit and for measuring a temperature of the liquid pharmaceutical
a controller disposed on an applicator of the plunger and coupled to the first electrode, the controller for measuring a linear displacement of the plunger within the barrel, the linear displacement derived from an electrical property of the first electrode alterable by an overlap of the first electrode by the second electrode, and coupled to the temperature sensor,
wherein the controller is in operable communication with a database for communication of a status of a liquid pharmaceutical contained within the smart syringe.

17. The smart syringe of claim 16, wherein the electrical property of the first electrode is inductance.

18. The smart syringe of claim 16, wherein the electrical property of the first electrode is capacitance.

19. The smart syringe of claim 16, wherein the second electrode is disposed on the barrel with an adhesive label.

20. The smart syringe of claim 19 further comprising an interface circuit coupled to the controller, the interface circuit for displaying information related to the liquid pharmaceutical to a user.
